(19) United States
(12) Patent Application Publication
(10) Pub. No.: US 2020/0352426 A1
(43) Pub. Date: Nov. 12, 2020

(54) CLEANING TOOL FOR ENDOSCOPE

(71) Applicant: OLYMPUS CORPORATION, Tokyo (JP)

(72) Inventors: Takuya TOYOOKA, Tokyo (JP); Tomokazu IWASAKI, Tokyo (JP)

(73) Assignee: OLYMPUS CORPORATION, Tokyo (JP)

(21) Appl. No.: 16/936,617

(22) Filed: Jul. 23, 2020

Related U.S. Application Data
(63) Continuation of application No. PCT/JP2018/027333, filed on Jul. 20, 2018.

(30) Foreign Application Priority Data
Jan. 31, 2018 (JP) 2018-015053

(51) Int. Cl. A61B 1/12; B08B 9/032

(52) U.S. Cl. CPC A61B 1/122; B08B 2209/032; B08B 9/032

(57) ABSTRACT

A cleaning tool for endoscope that cleans and disinfects a distal end constituent member provided in a distal end portion in an insertion section in an endoscope includes two components, and is configured to cover an entire periphery of the distal end constituent member by combining the two components, in which the two components are respectively provided with hinges configured to make the two components openable and closable by connecting the two components to each other, the hinge being configured to be separated from each other when an opening angle between the two components exceeds a predetermined angle.

### CROSS REFERENCE TO RELATED APPLICATION

This application is a continuation application of PCT/JP2018/027333 filed on Jul. 20, 2018 and claims benefit of Japanese Application No. 2018-015053 filed in Japan on Jan. 31, 2018, the entire contents of which are incorporated herein by this reference.

### BACKGROUND OF THE INVENTION

### 1. Field of the Invention

The present invention relates to a cleaning tool for endoscope that cleans and disinfects a distal end constituent member provided in a distal end portion in an insertion section in an endoscope.

### 2. Description of the Related Art

In recent years, diseased portions in a digestive tract system, a pancreaticobiliary system, and the like have been observed and treated using a so-called side-viewing endoscope (hereinafter merely referred to as an endoscope) in which an observation optical system is provided in a notch on an outer peripheral side surface of a distal end rigid member in a distal end portion positioned on a distal end side in an insertion direction (hereinafter merely referred to as a distal end side) of an insertion section to be inserted into a subject.

A configuration in which a site excluding the notch on the outer peripheral side surface of the distal end rigid member is covered with a cover has also been well-known.

In endoscopic treatment of a pancreatic duct, a biliary duct, a hepatic duct, or the like, the distal end portion in the insertion section in the endoscope is usually inserted into a vicinity of a duodenal papilla. Under X-ray fluoroscopy, a guide wire is made to protrude from a channel opening portion in a treatment instrument insertion channel provided within the insertion section and is inserted into the above-described duct.

Then, a method of selectively inserting a treatment instrument such as a catheter into the pancreatic duct, the biliary duct, or the hepatic duct using the guide wire as a guide has been used.

A configuration in which a raising base (forceps elevator) is provided at a position within the distal end portion facing the channel opening portion within the treatment instrument insertion channel has been well-known. The raising base varies a traveling direction of the guide wire or the treatment instrument inserted into the treatment instrument insertion channel toward the channel opening portion by being raised.

A configuration in which the guide wire or the treatment instrument is inserted into the above-described duct via the channel opening portion using the raising base has also been well-known.

The raising base is turnably supported on the distal end rigid member. The raising base is generally raised or lowered as the raising base turns by an operation wire (hereinafter merely referred to as a wire) being pulled or relaxed in the insertion direction by a raising base operation knob provided in an operation section in the endoscope. Note that the operation wire is inserted into the insertion section and a distal end in the insertion direction (hereinafter merely referred to as a distal end) of the operation wire is fixed to the raising base.

The side-viewing endoscope needs to be cleaned and disinfected to be reused after being used. However, the distal end rigid member is provided with many members each having irregularities such as the notch on the outer peripheral side surface, the raising base, and the channel opening portion, as described above.

Accordingly, to also reliably clean and disinfect the irregularities, cleaning and disinfection processing of a distal end constituent member such as the distal end rigid member, the observation optical system, the raising base, or the channel opening portion needs to be separately performed in addition to the normal cleaning and disinfection processing of the endoscope.

Japanese Patent Application Laid-Open Publication No. 2015-181914 discloses a configuration in which some of tubular members are inserted toward a raising base from a channel opening portion in a cleaning tool for endoscope that covers a distal end portion of an endoscope.

Further, Japanese Patent No. 6013658 discloses a configuration of a cleaning tool for endoscope that covers an entire periphery of a distal end portion of an endoscope and has a configuration in which a liquid can be supplied to a wide range including not only a raising base but also an outer surface of a distal end rigid member from a liquid supply port.

The configuration of the cleaning tool for endoscope disclosed in Japanese Patent No. 6013658 has a configuration in which a liquid supply port communicating with a liquid supply tube connected to the cleaning tool for endoscope is opened into the cleaning tool for endoscope.

### SUMMARY OF THE INVENTION

A cleaning tool for endoscope according to an aspect of the present invention is a cleaning tool for endoscope that cleans and disinfects a distal end constituent member provided in a distal end portion in an insertion section in an endoscope, the cleaning tool for endoscope including two components, and being configured to cover an entire periphery of the distal end constituent member by combining the two components, in which the two components are respectively provided with hinges configured to make the two components openable and closable by connecting the two components to each other, the hinges being configured to be separated from each other when an opening angle between the two components exceeds a predetermined angle.

A cleaning tool for endoscope according to another aspect of the present invention is a cleaning tool for endoscope that cleans and disinfects a distal end constituent member provided in a distal end portion in an insertion section in an endoscope, the cleaning tool for endoscope including two components, and being configured to cover an entire periphery of the distal end constituent member by combining the two components, in which the two components are configured to be separable in an up-down bending direction in a bending portion provided in the insertion section in the endoscope, and the two components are respectively provided with operation sections configured to be operated when the two components are separated from each other, and the operation sections are arranged, when the endoscope is placed on a plane such that the two components are combined with each other and a bending operation knob configured to bend the bending portion is oriented in a direction away from the plane, oriented in the direction away from the plane.

### BRIEF DESCRIPTION OF THE DRAWINGS

FIG. 1 is a diagram illustrating an endoscope in which a distal end constituent member provided in a distal end portion in an insertion section is cleaned and disinfected by a cleaning tool for endoscope according to the present embodiment;

FIG. 2 is a partial perspective view illustrating a cleaning and disinfection unit including the cleaning tool for endoscope that cleans and disinfects a distal end constituent member illustrated in FIG. 1 with two components in the cleaning tool for endoscope separated from each other;

FIG. 3 is a plan view illustrating a state where the distal end portion in the insertion section in the endoscope is placed on one of the components in the cleaning tool for endoscope illustrated in FIG. 2, as viewed in a III direction illustrated in FIG. 2;

FIG. 4 is a partial perspective view illustrating a state where the two components in the cleaning tool for endoscope are combined with each other from the placed state of the distal end portion illustrated in FIG. 3;

FIG. 5 is a diagram schematically illustrating a state where an operation section in an endoscope is placed on a plane in the combined state in the cleaning tool for endoscope illustrated in FIG. 4;

FIG. 6 is a cross-sectional view of the cleaning tool for endoscope and the distal end portion along a line VI-VI illustrated in FIG. 4; and

FIG. 7 is a cross-sectional view illustrating a state where one of the components in the cleaning tool for endoscope illustrated in FIG. 6 is opened from the other component.

### DETAILED DESCRIPTION OF THE PREFERRED EMBODIMENT

An embodiment of the present invention will be described below with reference to the drawings. It should be noted that drawings are schematic and a relationship between a thickness and a width of each of members, a ratio of respective thicknesses of the members, and the like respectively differ from actual ones, and it goes without saying that sections that differ in dimensional relationship or ratio may also be included among the drawings.

Note that a side-viewing endoscope will be described below as an example of an endoscope in the present embodiment.

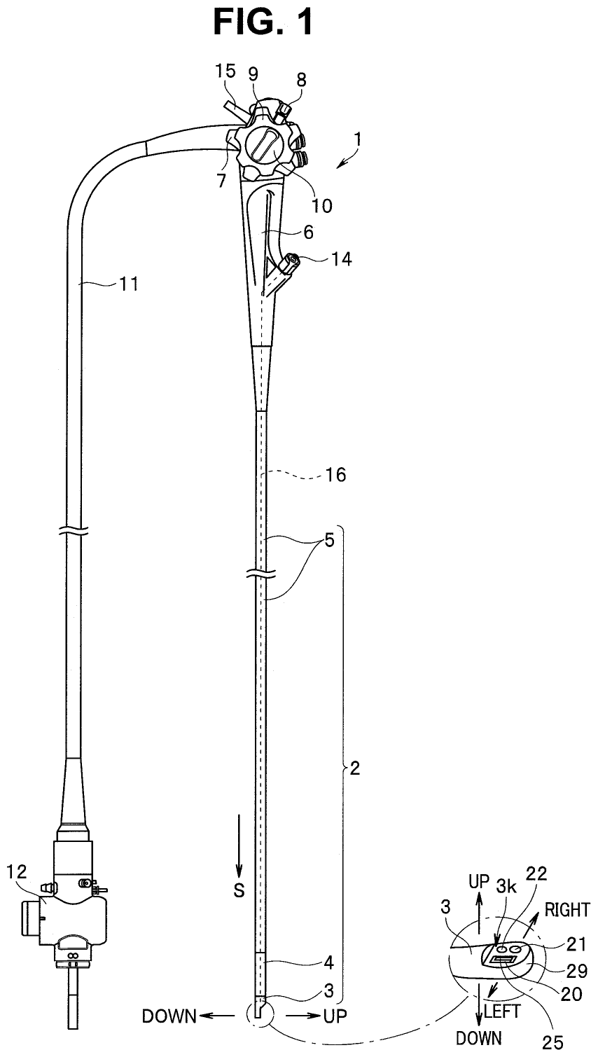

FIG. 1 is a diagram illustrating an endoscope in which a distal end constituent member provided in a distal end portion in an insertion section is cleaned and disinfected by a cleaning tool for endoscope according to the present embodiment.

As illustrated in FIG. 1, an endoscope 1 includes an insertion section 2 to be inserted into a subject, an operation section 6 consecutively provided on a proximal end side in an insertion direction S of the insertion section 2, a universal cord 11 extending from the operation section 6, and a connector 12 provided at an extension end of the universal cord 11, to constitute a main part.

The endoscope 1 is electrically connected to an external apparatus such as a control apparatus or an illumination apparatus via the connector 12.

The insertion section 2 is configured to include a distal end portion 3, a bending portion 4, and a flexible tube portion 5 in an order from a distal end side, and is formed in an elongated shape in the insertion direction S.

The operation section 6 is provided with an up-down bending operation knob 7 configured to bend the bending portion 4 in an up-down direction and a left-right bending operation knob 9 configured to bend the bending portion 4 in a left-right direction.

The operation section 6 is provided with a locking lever 8 configured to lock a turning position of the up-down bending operation knob 7 and a locking knob 10 configured to lock a turning position of the left-right bending operation knob 9.

The bending portion 4 is bent in four directions, for example, upward, downward, leftward, and rightward by a turning operation of the up-down bending operation knob 7 and the left-right bending operation knob 9, to make an observation direction of an image pickup unit not illustrated provided within the distal end portion 3 and improve insertability of the distal end portion 3 within the subject.

Note that as a bending direction of the bending portion 4, a bending direction toward a side on which an observation optical system 22, described below, is provided is defined as an up direction, and a direction opposite to the up direction is defined as a down direction.

Further, when the operation section 6 is placed on a plane F, a bending direction in a direction W away from the plane F is defined as a right direction, and a direction opposite to the right direction is defined as a left direction, as illustrated in FIG. 5, described below.

Further, the operation section 6 is provided with a raising base operation knob 15 configured to perform a turning operation when a raising base 25, described below, provided within the distal end portion 3 is raised or lowered.

The operation section 6 is provided with a treatment instrument insertion opening 14 configured to allow insertion of a guide wire or a treatment instrument not illustrated into a treatment instrument insertion channel 16 provided in the insertion direction S within the insertion section 2 in the endoscope 1.

A concave-channel-shaped notch portion 3k obtained by notching one outer peripheral surface side of a distal end rigid member 29 made of a metal, for example, as a distal end constituent member constituting the distal end portion 3 is formed on an outer peripheral surface of the distal end portion 3.

A channel opening portion 20 as an opening of the distal end portion 3 in the treatment instrument insertion channel 16 and a distal end constituent member is formed on one outer peripheral surface of the notch portion 3k.

An illumination lens 21 as a distal end constituent member and the observation optical system 22 are provided on the one outer peripheral surface of the notch portion 3k and in the vicinity of the channel opening portion 20.

Note that a distal end cover not illustrated covering the distal end rigid member 29, excluding the channel opening portion 20, the observation optical system 22, and the illumination lens 21, may be provided around the distal end rigid member 29.

Further, the raising base 25 as a distal end constituent member is provided at a position facing the channel opening portion 20 within the treatment instrument insertion channel 16 in the distal end rigid member 29.

The raising base 25 varies a traveling direction of a treatment instrument or a guide wire inserted into the treatment instrument insertion channel 16 from the treatment instrument insertion opening 14 toward the channel opening portion 20 side in the insertion direction S, to guide the treatment instrument or the guide wire to a desired position within the subject as the raising base 25 is raised.

The raising base 25 can be raised or lowered by pulling or relaxation in the insertion direction S of a wire not illustrated inserted into the insertion section 2 and the operation section 6 by a turning operation of the raising base operation knob 15.

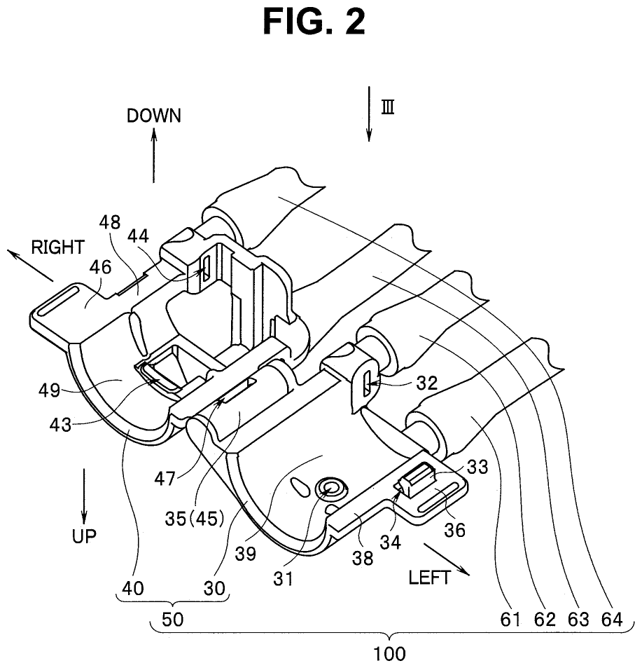

Then, a configuration of the cleaning tool for endoscope that cleans and disinfects the distal end constituent member described above will be described with reference to FIGS. 2 to 7.

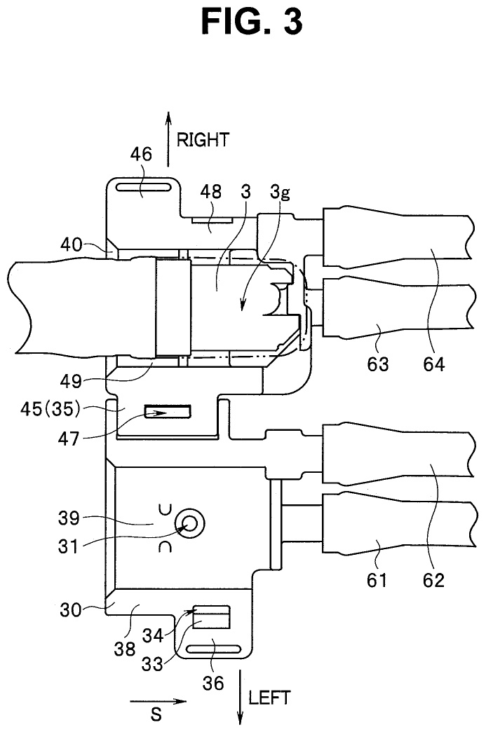

FIG. 2 is a partial perspective view illustrating a cleaning and disinfection unit including the cleaning tool for endoscope that cleans and disinfects the distal end constituent member illustrated in FIG. 1 with two components in the cleaning tool for endoscope separated from each other, and FIG. 3 is a plan view illustrating a state where the distal end portion in the insertion section in the endoscope is placed on one of the components in the cleaning tool for endoscope illustrated in FIG. 2, as viewed in a III direction illustrated in FIG. 2.

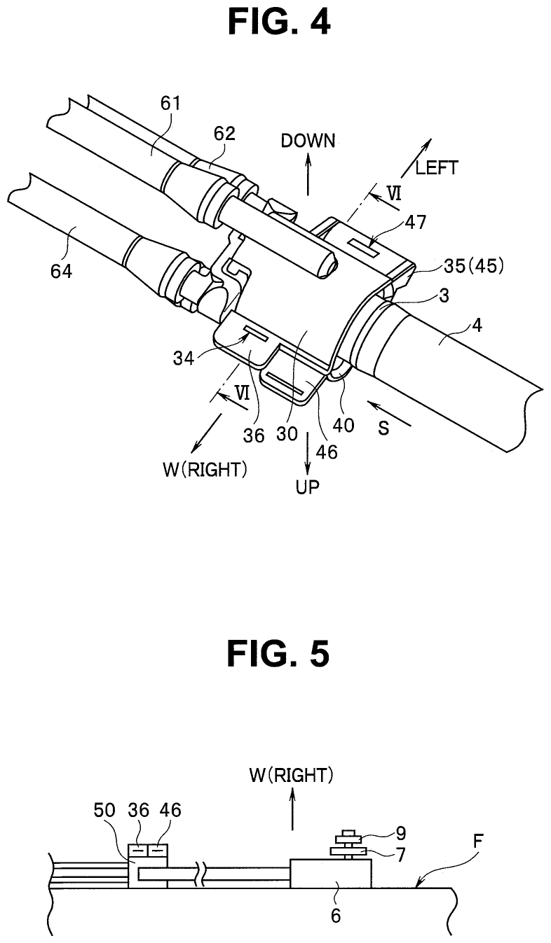

FIG. 4 is a partial perspective view illustrating a state where the two components in the cleaning tool for endoscope are combined with each other from the placed state of the distal end portion illustrated in FIG. 3, and FIG. 5 is a diagram schematically illustrating a state where the operation section in the endoscope is placed on the plane in the combined state in the cleaning tool for endoscope illustrated in FIG. 4.

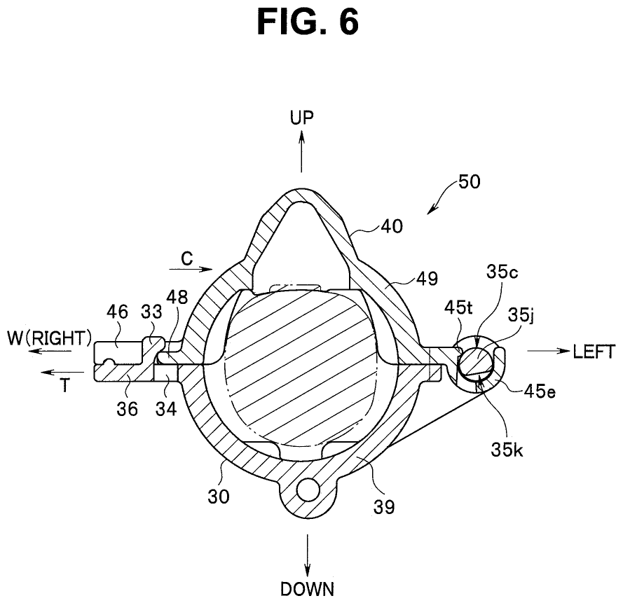

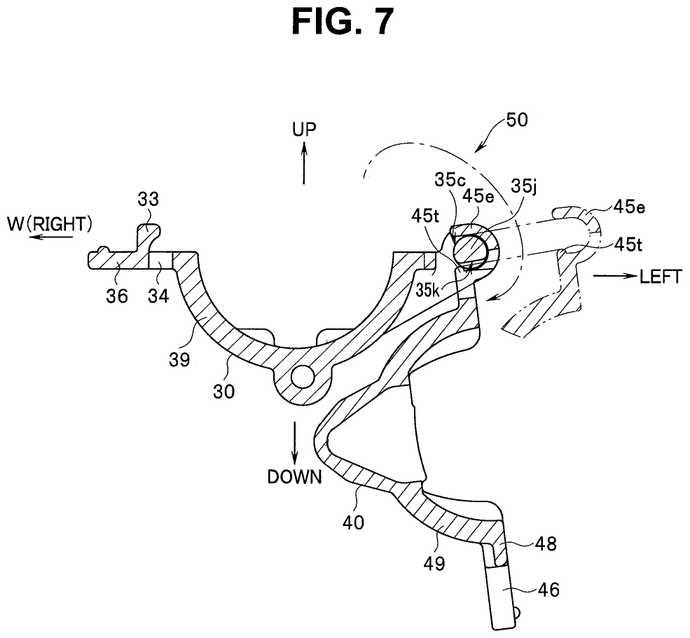

Further, FIG. 6 is a cross-sectional view of the cleaning tool for endoscope and the distal end portion along a line VI-VI illustrated in FIG. 4, and FIG. 7 is a cross-sectional view illustrating a state where one of the components in the cleaning tool for endoscope illustrated in FIG. 6 is opened from the other component.

In the following description, the distal end constituent member is indicated as the distal end portion 3 to simplify description. In other words, the distal end constituent member described as the distal end portion 3 as a cleaning and disinfection target of the cleaning tool for endoscope corresponds to all members provided in the distal end portion 3, e.g., the distal end rigid member 29, the channel opening portion 20, the illumination lens 21, the observation optical system 22, and the raising base 25, which were described above.

As illustrated in FIG. 2, a cleaning and disinfection unit 100 includes a cleaning tool for endoscope 50 and liquid supply tubes 61 to 64 connected to the cleaning tool for endoscope 50, to constitute a main part.

The cleaning tool for endoscope 50 includes two components 30 and 40 to be combinable and separable, as illustrated in FIGS. 2 to 4, 6, and 7.

The components 30 and 40 are configured to be formed into a cylindrical shape to cover an entire outer periphery 3g of the distal end portion 3 in a combined state, as illustrated in FIGS. 4 and 6, and to expose a part of the outer periphery 3g of the distal end portion 3 in a separated state, as illustrated in FIGS. 3 and 7.

More specifically, the components 30 and 40 are configured to be separable in an up-down bending direction of the bending portion 4, as illustrated in FIGS. 2 and 7.

More specifically, the components 30 and 40 are openably and closely connected to each other by hinges 35 and 45 respectively provided in the components 30 and 40, and are configured to be separable in the up-down bending direction by the hinges 35 and 45.

The hinge 35 includes a shaft 35j substantially parallel in the insertion direction S, as illustrated in FIGS. 6 and 7, and a circular arc portion 45e having a substantially semi-circular shape in cross section of the hinge 45 is fitted to an outer peripheral surface of the shaft 35j turnably and with movement regulated in the insertion direction S. As a result, the components 30 and 40 are configured to be operable and closable in the up-down bending direction by being connected turnably around the shaft 35j.

Accordingly, in the cleaning tool for endoscope 50, the components 30 and 40 are configured to be formed into a cylindrical shape to cover the entire outer periphery 3g of the distal end portion 3, as illustrated in FIGS. 4 and 6, in a closed state by the hinges 35 and 45, and to expose a part of the outer periphery 3g of the distal end portion 3 in an opened state, as illustrated in FIGS. 3 and 7.

The hinges 35 and 45 are difficult to clean and disinfect only by supplying a cleaning liquid and a disinfecting liquid because the circular arc portion 45e is fitted to the outer peripheral surface of the shaft 35j. Accordingly, a configuration in which the hinge 45 can be disassembled from the hinge 35 has been desired.

However, in a configuration in which the hinge 45 is simply disassembled from the hinge 35, the components 30 and 40 are simply separated from each other in an opened state. Thus, an attachment property of the component 40 to the component 30 decreases.

As illustrated in FIGS. 6 and 7, the hinges 35 and 45 are configured such that the components 30 and 40 are dissembled when an opening angle between the components 30 and 40 exceeds 260°. The opening angle is not limited to 260°. The components 30 and 40 may not come off each other unless the opening angle is 180° or more.

More specifically, in the hinges 35 and 45, a circular arc portion 35c and a notch 35k obtained by notching a part of the circular arc portion 35c are formed on the outer peripheral surface of the shaft 35j, as illustrated in FIGS. 6 and 7.

On one hand, the hinges 35 and 45 have a configuration in which a protrusion portion 45t formed on an inner peripheral surface of the circular arc portion 45e abuts on the circular are portion 35c with the components 30 and 40 closed, as illustrated in FIG. 6.

On the other hand, the hinges 35 and 45 have a configuration in which with the components 30 and 40 opened as the circular arc portion 45e rotates by 180° or more with respect to the shaft 35j, the circular arc portion 45e comes off the shaft 35j, as indicated by a two-dot and dash line illustrated in FIG. 7, when the abutment of the protrusion portion 45t on the circular arc portion 35c is released by the notch 35k, as illustrated in FIG. 7.

As a result, the components 30 and 40 have a configuration in which the component 40 is not dissembled from the component 30 in a merely opened state but is disassembled from the component 30 when opened by 180° or more.

An angle at which the hinges 35 and 45 in the components 30 and 40 can be disassembled may be an angle at which the components 30 and 40 are not unexpectedly separated from each other when the distal end portion 3 of the endoscope 1 is placed on the component 40 with the components 30 and 40 opened.

When the components 30 and 40 are separable at an angle less than 180°, for example, the components 30 and 40 may be separated from each other when an attempt to mount the distal end portion 3 of the endoscope 1 on the component 40 is made, resulting in deterioration in workability.

Note that the angle at which the components 30 and 40 are separated from each other can be freely set depending on a formation angle and a position of the notch 35k on the outer peripheral surface of the shaft 35j.

The liquid supply tubes 61 and 62 are connected, as illustrated in FIG. 2, to the component 30, and the component 30 is made of resin in white, for example. Note that the component 30 is made of resin in white to make it easy for an operator to visually recognize dirt that has adhered to the component 30.

The component 30 includes a semi-circular covering portion 39 covering the outer periphery of the distal end portion 3 and having a liquid supply port 31 in the liquid supply tube 61 formed on an inner peripheral surface of the semi-circular covering portion 39 and the hinge 35 extending in the right direction, for example, with the cleaning tool for endoscope 50 opened from an opening end of the covering portion 39. Further, the component 30 includes a flange 38 extending in the left direction, for example, and a liquid supply port 32 in the liquid supply tube 62 provided in the vicinity of the hinge 35.

Note that the liquid supply ports 31 and 32 are positioned facing a site which is not easily cleaned and disinfected, such as a movable portion such as the raising base 25 provided in the distal end portion 3, a turning axis of the raising base 25, a large recess provided in the distal end portion 3, and a recess for distal end cover attachment to the distal end rigid member 29 when the components 30 and 40 are closed and the entire periphery of the distal end portion 3 is covered with the components 30 and 40, as illustrated in FIG. 4.

Further, a knob 36 as an operation section extending in a left bending direction, for example, from the flange 38 with the cleaning tool for endoscope 50 opened is formed in the flange 38.

In the knob 36, a claw portion 33 raised from the knob 36 in a down bending direction, for example, and an opening portion 34 are formed with the cleaning tool for endoscope 50 opened.

The liquid supply tubes 63 and 64 are connected, as illustrated in FIG. 2, to the component 40. The component 40 is made of resin in a different color, for example, resin in green to make it easy to be distinguished from the component 30. A color of the component 40 may be any color if different from a color of the component 30.

The color of the component 40 also enables the cleaning tool for endoscope 50 to be assigned a specific meaning, for example, a possibility of specific cleaning processing (e.g., a possibility of autoclave processing if the color is green) and enables a user to recognize the specific meaning.

The component 40 includes a semi-circular covering portion 49 covering the outer periphery of the distal end portion 3 and having a liquid supply port 43 in the liquid supply tube 63 formed on an inner peripheral surface of the semi-circular covering portion 49 and the hinge 45 extending in the left direction, for example, with the cleaning tool for endoscope 50 opened from an opening end of the covering portion 49. Further, the component 40 includes a flange 48 extending in the right direction, for example, and a liquid supply port 44 in the liquid supply tube 64 provided in the vicinity of the flange 48. An opening portion 47 is formed in the hinge 45.

Note that the liquid supply ports 43 and 44 are positioned facing a site which is not easily cleaned and disinfected, such as a movable portion such as the raising base 25 provided in the distal end portion 3, a turning axis of the raising base 25, a large recess provided in the distal end portion 3, and a recess for distal end cover attachment to the distal end rigid member 29 when the components 30 and 40 are closed and the entire periphery of the distal end portion 3 is covered with the components 30 and 40, as illustrated in FIG. 4.

Further, a knob 46 as an operation section extending in a right bending direction, for example, from the flange 48 with the cleaning tool for endoscope 50 opened is formed in the flange 48.

Note that since the component 40 is configured in the color different from the color of the component 30, there is an effect of making it easy for the operator to understand, when the distal end portion 3 is placed on the cleaning tool for endoscope 50 in an opened state, a placement direction of the distal end portion 3, for example, placement of the distal end portion 3 in a direction in which the observation optical system 22 faces the covering portion 49 in the component 40, as illustrated in FIG. 3, for example.

Further, there is an effect of making the operator visually recognize from which of the liquid supply ports 43 and 44 and the liquid supply ports 31 and 32 a liquid is supplied first depending on a color and preventing a liquid supply order from being mistaken.

In addition, the components 30 and 40 are configured to be separable in the up-down bending direction of the bending portion 4, as illustrated in FIGS. 2 and 7, as described above. Thus, the operator easily recognizes the placement direction of the distal end portion 3 for the covering portion 39 in the component 30 or the covering portion 49 in the component 40.

As described above, the observation optical system 22 is provided in an up bending direction in the distal end portion 3. Therefore, if the distal end portion 3 is placed on the covering portion 39 or the covering portion 49 positioned in the up-down bending direction, with the cleaning tool for endoscope 50 opened, in such a manner that the observation optical system 22 faces the covering portion 39 or the covering portion 49, the placement direction of the distal end portion 3 is easily recognized.

The knobs 36 and 46 are operated to be grasped by the operator when a transition is made from a state where the cleaning tool for endoscope 50 is opened, as illustrated in FIGS. 2, 3, and 7, to a state where the cleaning tool for endoscope 50 is closed, as illustrated in FIGS. 4 and 6, or vice versa, that is, when the components 30 and 40 are opened or closed.

The knobs 36 and 46 are configured such that the flange 48 is snap-fitted to the claw portion 33 by elastically deforming the claw portion 33 in a closed state, as illustrated in FIGS. 4 and 6.

The knobs 36 and 46 are arranged in a direction oriented in a direction W away from the plane F when the operation section 6 is placed on the plane F such that the up-down bending operation knob 7 and the left-right bending operation knob 9 are oriented in the direction W, as illustrated in FIG. 5.

Usually, when the endoscope 1 is placed on the plane F, the operator places the operation section 6 on the plane F such that each of the operation knobs 7 and 9 is oriented in the direction W. Thus, the operation section 6 is placed with the up bending direction not matching the direction W.

When the direction in which the knobs 36 and 46 are arranged is made to match the right bending direction, the knobs 36 and 46 are arranged in a direction oriented in the direction W.

Accordingly, the operator easily performs an opening operation using the knobs 36 and 46 for the cleaning tool for endoscope 50 mounted on the distal end portion 3 in the endoscope 1 placed on the plane F.

Further, when the flange 48 to be snap-fitted to the claw portion 33 is formed integrally with the covering portion 49 for the component 40, the covering portion 49 is deformed to absorb movement of the flange 48 when the flange 48 is snap-fitted to the claw portion 33. Thus, the claw portion 33 need not be formed to be long and thin in the up bending direction.

When the components 30 and 40 are closed, the hinges 35 and 45 may be damaged by a stress load on the hinges 35 and 45 when the flange 48 extends beyond the claw portion 33. To prevent the hinges 35 and 45 from being damaged, the circular arc portion 45e to be fitted to the shaft 35j is formed in the component 40 to which a compressive force C is applied as the covering portion 49 is elastically deformed when the flange 48 extends beyond the claw portion 33.

As a result, when the flange 48 extends beyond the claw portion 33, as illustrated in FIG. 6, a pulling force T is not applied to the circular arc portion 45e. Thus, the circular arc portion 45e can be prevented from being damaged.

The opening portion 34 is an opening formed such that a snap fit portion of the flange 48 to the claw portion 33 is easily cleaned and disinfected, that is, a liquid is easily supplied to the snap fit portion. The opening portion 47 is an opening formed such that the hinges 35 and 45 are easily cleaned and disinfected, that is, a liquid is easily supplied to the hinges 35 and 45.

The other components are the same as the components in the conventional cleaning tool for endoscope.

Accordingly, in the present embodiment, the cleaning tool for endoscope 50 includes the two components 30 and 40 that are combinable, and is configured to cover the entire periphery of the distal end portion 3 by combining the components 30 and 40.

According to the configuration, a liquid can be supplied over a wide range to the distal end constituent member from the liquid supply ports 31, 32, 43, and 44, like in the conventional cleaning tool for endoscope that covers the entire periphery of the distal end portion 3. Further, the components 30 and 40 can be separated from each other, as illustrated in FIGS. 2 and 7. Thus, even when the liquid supply ports 31, 32, 43, and 44 and the liquid supply tubes 61 to 64 are clogged, the operator can easily confirm a clogging position by visual recognition after the separation.

From the foregoing, there can be provided a cleaning tool for endoscope 50 having a configuration in which the entire distal end constituent member provided in the distal end portion 3 in the insertion section 2 in the endoscope 1 can be reliably cleaned and disinfected without any defective liquid supply and a state of abnormal liquid supply is easily confirmed.

Note that although a case where the components 30 and 40 are separable and combinable by being openable and closable while turning by the hinges 35 and 45 has been illustrated as an example in the present embodiment described above, the present invention is not limited to the component. The components 30 and 40 may be separable by the other components.

Although the side-viewing endoscope has been described as an example of an endoscope in the above-described embodiment, the present invention is not limited to the side-viewing endoscope. The cleaning tool for endoscope 50 is also applicable to a direct-viewing endoscope in which a treatment instrument insertion channel is provided with a raising base.

Further, the present invention is not limited to an endoscope, and it goes without saying that the cleaning tool for endoscope 50 is also applicable to a treatment instrument in which a distal end portion in an insertion section such as a catheter is provided with a raising base.

What is claimed is:

1. A cleaning tool for endoscope that cleans and disinfects a distal end constituent member provided in a distal end portion in an insertion section in an endoscope,
the cleaning tool for endoscope comprising two components, and being configured to cover an entire periphery of the distal end constituent member by combining the two components,
wherein the two components are respectively provided with hinges configured to make the two components openable and closable by connecting the two components to each other, the hinges being configured to be separated from each other when an opening angle between the two components exceeds a predetermined angle.

2. The cleaning tool for endoscope according to claim 1, wherein the predetermined angle is 180°.

3. The cleaning tool for endoscope according to claim 1, wherein the two components are configured to be formed into a cylindrical shape covering the distal end constituent member in a combined state and to expose a part of an outer periphery of the distal end constituent member in a separated state.

4. The cleaning tool for endoscope according to claim 1, wherein the two components are configured to be separable in an up-down bending direction in a bending portion provided in the insertion section in the endoscope.

5. The cleaning tool for endoscope according to claim 4, wherein
the two components are respectively provided with operation sections configured to be operated when the two components are separated from each other, and
when the endoscope is placed on a plane in a state where the two components are combined with each other to cover the entire periphery of the distal end constituent member by the two components, such that a bending operation knob configured to bend the bending portion is oriented in a direction away from the plane, the operation sections are arranged to orient in the direction away from the plane.

6. The cleaning tool for endoscope according to claim 1, wherein the hinge includes a shaft substantially parallel in an insertion direction of the insertion section in the endoscope, and turnably connects the two components around the shaft.

7. The cleaning tool for endoscope according to claim 1, wherein the two components are configured to be formed into a cylindrical shape covering the distal end constituent member in a closed state and to expose a part of an outer periphery of the distal end constituent member in an opened state.

8. The cleaning tool for endoscope according to claim 1, wherein the two components are respectively made of materials having different colors.

9. The cleaning tool for endoscope according to claim 1, wherein the two components are each made of resin.

10. A cleaning tool for endoscope that cleans and disinfects a distal end constituent member provided in a distal end portion in an insertion section in an endoscope,
the cleaning tool for endoscope comprising two components, and being configured to cover an entire periphery of the distal end constituent member by combining the two components, wherein
the two components are configured to be separable in an up-down bending direction in a bending portion provided in the insertion section in the endoscope, and
the two components are respectively provided with operation sections configured to be operated when the two components are separated from each other, and the operation sections are arranged, when the endoscope is placed on a plane such that the two components are combined with each other and a bending operation knob configured to bend the bending portion is oriented in a direction away from the plane, oriented in the direction away from the plane.
